(19) United States
(12) Patent Application Publication
(10) Pub. No.: US 2005/0142229 A1
(43) Pub. Date: Jun. 30, 2005

(54) Pharmaceutical activity and manufacturing method of the four-plant-derived coronary blood flow enhancing agent

(76) Inventors: Jiao Gong, Arcadia, CA (US); Wei Jiang, Arcadia, CA (US)

Correspondence Address: JIAO GONG, 411 S. 3RD AVE #B, ARCADIA, CA, 91006, US

(21) Appl. No.: 10/745,811

(22) Filed: Dec. 29, 2003

(51) Int. Cl. A61K 35/78

(52) U.S. Cl. 424/746; 424/773; 424/774

(57) ABSTRACT

Nitroglycerine, the classic drug for myocardial ischemia, has been used over a century. By providing exogenous No., it alleviates the myocardial ischemia and is mainly used in the first aid of myocardial ischemia. The 4-Medicinal-Plant-Derived Coronary Blood Flow enhancing Agent is the result of strictly controlled processing procedures including the followings: extracting with Carbon Dioxide Supercritical Fluid; WLD resin adsorbing, eluting, separating and purifying; high performance liquid chromatography and fingerprint analysis to ensure the balanced and stable quality. Pharmacological experiments indicated that it increased coronary blood flow, lowered peripheral vascular resistance, regulated myocardial oxygen supply-demand balance and decreased the oxygen consumption index as well as myocardial oxygen consumption. It takes effect in a way different from Nitroglycerine. This agent can be used for the prevention and treatment of myocardial ischemia, coronary heart disease and angina pectoris.

### BACKGROUND OF THE INVENTION

“Pharmacological Activity & Manufacturing Method of 4-Medicinal-Plant-Derived Coronary Blood Flow Enhancing Agent” can be grouped into the US combined patent on pharmacology, isolation and purification of medicinal plants. This agent can increase coronary blood flow, decrease heart rate, low blood pressure and left ventricular pressure, reduce oxygen consumption index and myocardial oxygen consumption in the isolated guinea pig heart perfusion model.

According to the Examination Guideline of US patent & Trademark Office, data from in vitro or animal experiment, in general, are sufficient to support the therapeutic practicality. If they are reasonably associated with specific therapy or pharmacological practicality, data from in vitro, animal or combined experiment are absolutely sufficient to determine the practicality of a certain compound, synthesized chemical or processing method. The evidences needn't be obtained from experiments with well-accepted related animal model. The experimental data, if they have reasonable relationship with the practicality described in the patent application, should be evaluated substantially. So, the data provided by the applier can be experimental report based on a specific animal model and combined with the appropriate explanation of the practicality supported by experiments. If technicians believe that the conclusion from animal experiment has reasonable prediction in the human practicality, considerable thinking should be given to the evidences from experiments which support the practicality. The Pharmacological Activity & Manufacturing Method of 4-Medicinal-Plant-Derived Coronary Blood Flow Enhancing Agent is consistent with the rules of patentability mentioned above. Physiologically, coronary artery supplies the myocardium with blood and oxygen. Although the heart only account fro 0.5% of the body weight, but the coronary blood flow volume is as high as 5% of the cardiac output. Not only the myocardial blood flow but also the blood oxygen uptake rate is high In the normal condition, the arterial oxygen content is 20%, but the venous oxygen content in the coronary sinus is only 7%, indicating that 70% oxygen was uptaken by the myocardium which is three times as high as that of the other organ (22%). It is because the aerobic metabolism predominates in the myocardium. Coronary blood flow is determined by myocardial oxygen consumption, so the determinant factors of myocardial oxygen consumption, so the determinant factors of myocardial oxygen consumption also affects coronary blood flow. Normally speaking, if the heart beats stronger and faster, it needs more oxygen. So, the coronary blood flow will increase correspondingly to meet the increased oxygen demand. The major etiologic factor of myocardial ischemia or coronary heart disease is atherosclerosis which is related to low density lipoprotein, hyperhomocysteinemia, estrogen, and myocardial infarction. The classic drug for myocardial ischmia is nitroglycerine, which takes effect through providing exogenous nitrogen oxide (NO). The 4-Medicinal-Plant-Derived Coronary Blood Flow Enhancing Agent is more suitable for the prevention and preventive therapy of myocardial ischemia.

### BRIEF SUMMARY OF THE INVENTION

Both the left and right coronary artery originate from the aoritic root and supply the myocardium with nutrient and oxygen. In the isolated heart perfusion experiment, the myocardium was perfused with oxygenated, thermostatic and pressure-constant KH solution via the coronary artery connected with aortic cannula; and the coronary venous effluence was collected for the coronary blood flow determination. Pharmacological experiment indicated that the 4-Medicinal-Plant-Derived Coronary Blood Flow Enhancing Agent increased the coronary blood flow in the isolated guinea pig heart. Isoprenaline obviously increased heart rate and myocardial contraction although it enhanced the coronary blood flow.

In the pharmacological experiment, the hemodynamic and coronary blood flow of the anaesthetized canine was studied by Eight Channel Physiological Recorder. The results indicated that this agent slowed down the heart rate, lowered the blood pressure, decreased the left ventricular pressure and reduced the oxygen consumption index as well as the myocardial oxygen consumption. This agent also protected the ischemic myocardium and reduced the myocardial ischemia degree by lowering the peripheral vascular resistance, regulating the oxygen supply and demand balance, decreasing the myocardial tension and improving the blood supply of the endocardium. In addition, this agent improved the cardiac function and hemodynamics by dilating coronary artery, increasing coronary blood flow, decreasing myocardial oxygen consumption, regulating myocardial metabolism and lowering the peripheral vascular resistance. This agent is useful in the prevention and preventive therapy of myocardial ischemia, coronary heart disease and angina pectoris. (Table 3).

### BRIEF DESCRIPTION OF THE SEVERAL VIEWS OF THE DRAWING

TABLE 1
Raw Material Source & Formula of the 4-Medicinal-Plant-Derived
Coronary Blood Flow Enhancing Agent

| | | |
|---|---|---|
| 1. | Rhizoma Chunanxiong | 20% |
| | Unbelliferae | |
| | Root and Stem of Ligusticum Chuanxiong Hort. | |
| 2. | Radix Salviae Miltiorrhizae | 33% |
| | Labiatae | |
| | Root and Stem of Salvia Miltiorrhiza Bge. | |
| 3. | Fios Carthami | 20% |
| | Compositae | |
| | Flower of Corthamus TincoriusL. | |
| 4. | Rhizoma Corydalis | 27% |
| | Papaveraceae | |
| | Stem Tuber of Corydalis Yanhusuo W. T. Wang | |
| | Total | 100% |

TABLE 2
Manufacturing Method and Flow Chart of the 4-Medicinal-Plant-Derived
Coronary Blood Flow Enhancing Agent

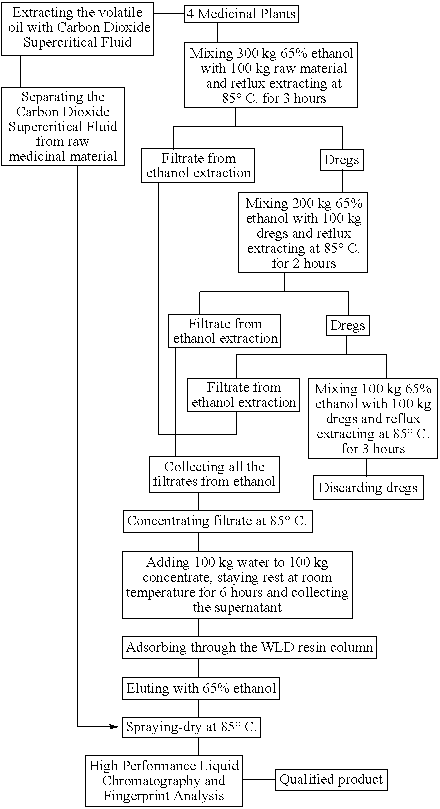

Part III Pharmacological Activity of 4-Medicinal-Plant-Derived Coronary Blood Flow Enhancing Agent

1. Effect on coronary blood flow of the isolated guinea pig heart.
(1) The perfusion circuit was maintained at 37±1° C. and the perfusate in the condensing tube was thermostatic. The upper end of the condensing tube was connected with reservoir and the lower with aortic cannula via rubber tube. The perfusate level in reservoir A was constant by controlling the height of liquid below the air inlet in reservoir B. The perfusate level was about 50 cm above the aortic root. Constant oxygen was gassed into reservoir A to make the KH solution saturated with oxygen. When full of the oxygenated LH solution, the perfusion tube was clamped.
(2) On rat, weighing 300 g, was stunned and exsanguinated via carotid. The chest and pericardium were opened, and then the heart was suspended in the pericardial cradle. The heart was exercised and immediately put into the cold KH solution (4° C.). The remaining blood was pressed out gently. After aortic cannulation at the top of the ascending aorta, the clamp was opened and the coronary venous effluent was present (route: coronary artery→myocardium→right atrium→vena cava and pulmonary artery). The pressure transducer was connected with the apex of heart to record the graphs and count the heart beat. The coronary venous effluent dripped into the measuring cup via a funnel.
(3) Coronary Blood Flow Measurement: The heart was allowed to recover for 10 minutes to stabilize coronary blood flow, heart rate, and heart beat amplitude. The per-minute flow volume was measured consecutively for 3 minutes. If the values were similar, the average was calculated for the normal coronary blood flow. For guinea pig, the value was about 10 ml/min, which was regulated by perfusion pressure in accordance with the heart size.
(4) Effect Observation Effect after Administration: The test sample and isoprenaline was injected into the aortic cannula respectively. The per-minute coronary blood flow, heart rate and heart beat duration were measured during 1˜10 min after administration, the maximum and minimum were picked out to caculate the extreme difference. Note: the second administration should not be given until the coronary blood flow returned to normal. Five replicate experiments were conducted to ensure the reproductivity.

Results: This agent increased the coronary blood flow of the isolated guinea pig heart. Although it increased the coronary blood flow, isoprenaline increased the heart rate and myocardial contraction. (Table 1)

TABLE 1
Effect of Test Sample and Isoprenaline on the Coronary Blood Flow, Heart Rate and Heart Beat Duration
(X ± SD)

| | | Coronary Blood Flow | | Heart Rate | | Heart beat duration | |
|---|---|---|---|---|---|---|---|
| | | (ml/min) | | (time/min) | | (min) | |
| | | Before | After | Before | After | Before | After |
| Drug Name | Dosage | administration | administration | administration | administration | administration | administration |
| Test Sample | 0.15 g | 13 ± 2 | 21 ± 5** | 140 ± 16 | 117 ± 17*** | 20 ± 3 | 21 ± 4* |
| Test Sample | 0.15 g | 13 ± 4 | 23 ± 6*** | 145 ± 12 | 116 ± 15** | 20 ± 4 | 21 ± 5* |
| Isoprenaline | 0.25 μg | 14 ± 5 | 20 ± 4*** | 142 ± 18 | 198 ± 21*** | 21 ± 3 | 62 ± 10*** |

2. Effect on the Hemodynamics and Coronary Blood Flow of Anaesthetized Canine. Dogs, weighing 15˜20 kg, were anaesthetized by intravenous injection of sodium pentobarbital (30 mg/kg), and fixed on the operating table in the right lateral decubitus position. The preparations included: Shaving the neck, chest an inner hind leg; isolating femoral vein and inserting venous cannula; mounting the microsyringe (first slowly transfusing normal saline); isolating trachea for artificial ventilation; seperating carotid and threading two suturing thread under the carotid for cannulation. The thoracectomy was performed along the 4˜5 intercostal space and the heart was exposed. Positive artificial ventilation was started (15˜20 cycles/min). Pericardium was opened longitudinally to make the “pericardial cradle”. On the top of the isolated aortic root and left coronary anterior descending branch (or left circumflex branch), the electromagnetic flowmeter probe was placed to measure cardiac output and coronary blood flow (10˜15 mm calibred probe for aorta; 1.5˜3 mm calibred probe for coronary artery.). The heparinized-perftisate-filled catheters were inserted into common carotid and left ventricle to measure the blood pressure and intraventricular pressure respectively, 5 mg/kg heparin was injected via femoral vein though T-Value. The ECG electrode was inserted under the skin of the extremities to record the lead II electrocardiograph and count the heart rate. The electrical signal of the left intraventricular pressure was input into the electrical differential analyzer to measure the left intraventricular pressure change rate. All date were input into the Eight Channel Physiological Recorder Simultaneously.

The normal graph was recorded first, and then recorded again intravenous injection of test sample via femoral vein. (See Table 2)

All the data mentioned above were primary parameters, from which the second parameters of hemodynamics were deduced.

Results: This agent slowed down the normal animals heart rate, lowered blood pressure as well as left intraventricular pressure, and decreased the oxygen consumption index and myocardial oxygen consumption.

TABLE 2
Effect on the hemodynamics and coronary blood flow in the canine mode (x ± SD)

| | | After | |
|---|---|---|---|
| | Before | administration | Change rate |
| Item | administration | (5 min) | (%) |
| Heart rate (beat/min) | 151 ± 27 | 128 ± 28 | −15.67 ± 3.95 |
| Mean arterial pressure (kPa) | 14.26 ± 4.27 | 10.26 ± 5.47 | −20.13 ± 27.69 |
| (mmHg) | (107 ± 32) | (77 ± 41) | |
| Left ventricular pressure, LVP (kPa) | 6.93 ± 3.20 | 6.27 ± 4.00 | −15.47 ± 29.77 |
| (mmHg) | (52 ± 24) | (47 ± 30) | |
| Maximum LVP increase (kPa/s) | 135.57 ± 44.39 | 102.24 ± 60.52 | −17.27 ± 27.97 |
| (mmHg/s) | 1017 ± 333 | 767 ± 454 | |
| Cardiac output (L/min) | 0.62 ± 0.30 | 0.43 ± 0.36 | −28.83 ± 24.39 |
| Stroke volume (ml/cycle) | 3.96 ± 1.37 | 3.08 ± 2.00 | −17.97 ± 26.49 |
| Cardiac index (L/min · m²) | 1.02 ± 0.48 | 0.67 ± 0.53 | −28.83 ± 24.39 |
| Stroke index (ml/cycle · m²) | 6.57 ± 2.37 | 4.91 ± 2.86 | −17.97 ± 26.49 |
| Cardiac work index (kg · M/ | 16.10 ± 9.94 | 8.84 ± 10.19 | −29.63 ± 32.78 |
| min · m²) | | | |
| Total peripheral resistance (kPa · s/L) | 1538.9 ± 528.8 | 1706.6 ± 558.0 | 11.50 ± 1.72 |
| Coronary blood flow (ml/min) | 50.67 ± 8.96 | 45.00 ± 2.00 | −9.83 ± 6.85 |

### Detailed Description of the Invention

1. “The Pharmacological Activity & Manufacturing Method of the 4-Medicinal-Plant-Derived Coronary Blood Flow Enhancing Agent” can be grouped in the US combined patent on pharmacology, isolation and purification of medicinal plant. The formula consists of Rhizoma Chuanxiong 20%, Radix Salviae Miltiorrhizae 33%, Fios Carthami 20%, Rhizoma Corydalis 27%.

2. The processing method can be described as follows: extracting with Carbon Dioxide supercritical fluid and separating the extractant for rectification; Mixing 300 kg, 200 kg, 100 kg 65% ethanol with 100 kg raw material and reflux extracting at 85° C. for 3, 2, 1 hours respectively; Concentrating the extractant and adding 100 kg water to 100 kg concentrate; staying rest for sendimentation at room temperature for 6 hours; collecting supernatant and adsorbing with WLD resin column; eluting with ethanol; collecting elution and extractant of carbon Dioxide Supercritical fluid extraction; Spraying-dry at 85° C.; high-performance liquid chromatography and fingerprint analysis to ensure the quality.

3. Pharmacological experiments indicated that this agent increased the coronary blood flow of the isolated guinea pig heart. The further experiment was conducted to study the hemodynamics and coronary blood flow in the anaesthetized canine model, and the results indicated this agent slowed down heart rate, lowered blood pressure, decreased left intraventricular pressure and reduced the oxygen consumption index oxygen consumption.

1. I claim that “The Pharmacological Activity & Manufacturing Method of the 4-Medicinal-Plant-Derived Coronary Blood Flow enhancing Method” is my invention. The four medicinal plants are Rhizoma Chuanxiong, Radix Salviae Miltiorrhizae, Fios Carthami, Rhizoma Corydalis.
I believe my invention includes the following: extracting with Carbon Dioxide Supercritical fluid for rectification; extracting with ethanol and processing with water; adsorbing with WLD resin column and then eluting, isolating, and purifying; colleting the two extractant (carbon dioxide & ethanol) and spraying-dry; qualitification and quantitative analysis:
I believe that the 4-Medicinal-Plant-Derived Coronary Blood Flow Enhancing Agent can increase coronary blood flow, lower the peripheral vascular resistance, regulate the oxygen supply-demand balance, and decrease oxygen consumption index as well as myocardial oxygen consumption. It can be used in the prevention and treatment of myocardial ischemia and coronary heart disease.
